(19) United States
(12) Patent Application Publication
(10) Pub. No.: US 2006/0183111 A1
(43) Pub. Date: Aug. 17, 2006

(54) Primary human hepatocytes for native hepatitis C virus (HCV) replication

(76) Inventors: Ajit Kumar, Bethesda, MD (US); Krishna Banaudha, Rockville, MD (US)

Correspondence Address: ANTONELLI, TERRY, STOUT & KRAUS, LLP, 1300 NORTH SEVENTEENTH STREET, SUITE 1800, ARLINGTON, VA, 22209-3873, US

(21) Appl. No.: 11/334,392

(22) Filed: Jan. 19, 2006

Related U.S. Application Data
(60) Provisional application No. 60/653,501, filed on Feb. 17, 2005.

(51) Int. Cl. C12Q 1/70; C12N 5/08

(52) U.S. Cl. 435/5; 435/370

(57) ABSTRACT

Novel methods are disclosed for the long-term culture of human primary hepatocytes in tissue culture system. This culture system supports the replication, gene expression and dissemination of a multitude of pathogenic agents that reside in, or pass through, the liver. Among these, the invention advantageously provides for efficient hepatitis C virus (HCV) replication, and dissemination. Additionally disclosed are methods for the determining cellular receptors, hepatocyte cell subtypes, and host cell molecules required for efficient hepatic pathogen replication, expression and dissemination.

This application claims priority to Provisional Application No. 60/653,501 filed Feb. 17, 2005.

### GOVERNMENT INTEREST

This invention was made, in part, with U.S. government support under National Institutes of Health grant number AI 054222. The U.S. government has certain rights in the invention.

### BACKGROUND OF THE INVENTION

1. Technical Field of the Invention

The present invention is related to the establishment of novel, long-term human primary hepatocyte cell culture systems capable of sustaining the replication, expression and dissemination of hepatotrophic and other viruses, particularly (but not limited to) human hepatitis C virus (HCV), and other pathogens, as well as useful methods that advantageously employ such primary hepatocyte cell culture systems. Primary human hepatocytes for cultivation in hormonally defined medium are purified from donor tissue by liver perfusion techniques.

2. Description of the Related Art

Hepatitis C virus (HCV) is the etiologic agent for non-A non-B hepatitis, which affects nearly 200 million people worldwide. HCV infected individuals may remain asymptomatic, or may develop chronic hepatitis or cirrhosis that can lead to hepatocellular carcinoma (Alter, H. J. & Seef, L. B. 2000). The genetic parameters, both in the virus and the host, giving rise to these differing outcomes are not currently well understood at the molecular level.

Currently, there is no vaccine for HCV. Known drug therapies for HCV are not well tolerated by most patients, and thus, only a small percentage of patients benefit from these drug therapies. A major obstacle preventing drug manufacturers from developing vaccines or drugs to treat HCV is the inability of scientists to propagate native infectious forms of the virus in tissue culture system. A long term cultures of human primary hepatocytes that elaborate markers of normal liver differentiation is essential to sustain native virus replication.

Low levels of replicating forms of HCV RNA in hepatocytes have been reported (Boisvert et al., 2001). In this study the investigators isolated blood and liver cells from HCV infected patients and show virus replication in the infected patients. The investigators (Boisvert et al., 2001) do not disclose a culture system for sustained virus replication. There have also been several studies based on pseudotyped virion; these are surrogate system with the outside shell of HCV and the replication machinery of another virus, usually HIV. Therefore, the system is good only to test how the outer shell of HCV behaves in an artificially replicating system. It is not a system of replication for native virus. These studies only describe crucial aspects of viral attachment and dissemination instead of a replicating system for native virus. (Cormier, et al., 2004; 2004a; Flint et al., 2004; Lozach, et al., 2004; Ludwig, et al., 2004; Moradpour, et al., 2004).

Several cellular proteins have been proposed as candidate receptors for HCV including CD81 and the low density lipoprotein receptor (Pileri et al., 1998; Agnello et al., 1999). Recent reports suggest that CD81 functions as HCV post-attachment entry co-receptor, leaving room for other proteins that might function in viral dissemination (Cormier, et al., 2004a; Zhang et al., 2004). Studies on HCV entry, based on pseudotyped virus bearing HCV glycoproteins E1 E2, show restricted tropism to human liver cell lines. Although all the permissive cells studied expressed CD81, expression of CD81 does not appear to be sufficient for viral entry (Zhang et al., 2004).

The HCV envelope is formed by two heavily N-glycosylated type 1 transmembrane glycoproteins E1 (31 kDa) and E2 (70 kDa) that are expressed as heterodimers on viral surface (Lagging et al., 1998). The C-type lectins (DC-SIGN and L-SIGN) have been shown to bind envelope glycoproteins from a number of viruses including HIV-1, and Ebola (Geijtnbeek, et al., 2000; Alvarez et al., 2002). DC-SIGN (dendritic cell specified intracellular adhesion molecule-3-grabbing nonintegrin, CD209), and L-SIGN (DC-SIGNR, liver and lymph node-specific, CD209L), are known to be expressed in liver tissue by Kupffer cells and the sinusoidal endothelial cells (LSEC) respectively. Recent studies utilizing pseudotyped HIV/HCV particles show that the C-type lectins function to capture and transmit HCV (Cormier et al., 2004; Flint et al., 2004; Lozach et al., 2004; Ludwig et al., 2004). Depending on the cell type, HCV targets DC-SIGN and L-SIGN to escape lysosomal degradation.

However, it remains unclear whether HCV infection of hepatocytes depends on its interaction with CD81 co-receptor, and whether HCV dissemination in infected liver is regulated by the C-type lectins, DC-SIGN and L-SIGN, or additional cellular factors because none of the previous studies were done with native infectious virus. A human primary liver culture that efficiently replicates and disseminates HCV would significantly aid the basic understanding of chronic hepatitis and hepatocellular carcinoma.

The persistence of HCV infection and chronic hepatitis suggests that the virus has developed mechanisms to escape host immune response. A system for native HCV replication in primary hepatocytes would greatly facilitate analysis of basic parameters of viral infection, such as how HCV specifically targets and persists in liver cells. Additionally, such a system would be useful for determining whether an alternate receptor or “capture receptors” play a significant role in the infection and dissemination of HCV in human liver. Importantly, our primary hepatocyte culture system lays the foundation for ex vivo analysis of the regulation of virus propagation in biopsy tissues obtained from HCV infected patients during routine diagnosis. Such a direct evaluation of the contributions of parenchymal and nonparenchymal liver cells in the development of HCV-mediated chronic liver disease and hepatocellular carcinoma is not possible by current procedures of HCV replication in hepatoma cell lines.

Hepatitis C virus establishes chronic infection in up to 75% of those infected by evading virus-specific immunity. A suitable culture system to propagate infectious clones of HCV that are predominant in the United States, and which displays properties of natural HCV infection, is important to study virus-host interactions crucial for developing effective strategies to control HCV. Most full-length viral constructs containing cell culture-adaptive mutations do not produce infectious particle; the exception being JFH-1 isolate from a Japanese fulminant hepatitis patient. Recently, three groups (Lindenbach et al., 2005; Wakita et al., 2005; Zhong et al., 2005) reported successful replication of infectious virion utilizing full length clones of JFH-1 in Huh-7.5 (human hepatoma) cell line. Permissiveness of Huh-7.5 cells is considered to be due to inactivating mutation in RIG-I gene, an essential regulator for ds-RNA-induced signaling which is crucial in host response to HCV infection (Sumpter et al., 2005; Yoneyama et al., 2004).

A full-length HCV chimera of genotype 2a (FL-J6/JFH) which efficiently replicates and produces infectious virus particles in Huh-7.5 cell line has been reported (Lindenbach et al., 2005). Lindenbach et al. also showed that full length clones of HCV JFH-1 and genotype 1a (FL-H77/JFH) was not capable of spreading infection in Huh-7.5 cell line. The cell line used by Lindenbach et al., the Huh-7.5, a human hepatoma derived cell line, is rendered more permissive for virus replication due to the inactivating mutation in RIG-I gene which is the ‘gate keeper’ that senses infection. Therefore, although the hepatoma cell line (Huh-7.5) is suitable for the replication of select HCV clones (as described by Lindenbach et al., 2005), it is not suitable for the propagation of native HCV strains that commonly infect vast majority of HCV patients world wide.

Sumpter et al., 2005 (discloses) reported that RIG-I gene function is crucial in the processes that trigger host defense against viral infection. This response is absent in cells selected for permissiveness to HCV replication, such as the Huh-7.5 cell line; which makes the Huh-7.5 cell line less suitable for screening of candidate drugs for antiviral therapy and vaccine against HCV infection.

Yoneyama et al., 2004 reported the identification of RIG-I gene which is essential for regulating dsRNA (double stranded RNA) signals in virus infected cells. In virus infected cells double stranded RNA accumulates (as a normal process of virus replication). Host cells have developed molecular mechanisms for the detection of dsRNA as a means of stimulating their antiviral defenses. Yoneyama et al. describe the RIG-I gene as the key element in detecting the dsRNA signaling in virus infected cells. In their studies, Yoneyama et al. compared the functions of RIG-I gene in primary cells which have normal RIG-I, and cell lines which have mutation in RIG-I gene. Yoneyama et al. disclose cell lines that suffer inactivating mutation in RIG-I gene that will be naturally permissive to HCV replication, whereas primary hepatocyte cultures with normal RIG-I function will elaborate native antiviral response. Yoneyama et al., 2004 do not disclose a system of native HCV propagation that is optimally suited for the development of antiviral measures.

Hence, there is a need for a primary hepatocyte culture system that allows native HCV strains to propagate, and shows sensitivity to interferon treatment similar to the experience of the HCV infected patients. It is important to note that the Huh-7.5 cells which lack RIG-I gene function, will not be suitable for the evaluation of host response to native HCV infection, which is critically dependent on RIG-I (and related human genes of antiviral defense) mediated sensing of viral infection. The inventors of this invention have invented a primary hepatocyte culture system for native HCV strains, which is the only suitable system for evaluating candidate drugs for antiviral therapy and the vaccine against HCV infection.

The invention utilizes a system of tracking functional HCV replication complexes in human primary hepatocytes (HCV subgenomic replicons for this study were kindly provided by Dr. Charles Rice, The Rockefeller University). The culture system of this invention supports the replication and propagation of infectious HCV clones of genotypes 1a and 1b, and 2a in human primary liver cells. The key features of our primary human hepatocyte based culture system for the replication of HCV that distinguishes it from all the published reports are:
(a) The primary human hepatocyte cultures (HHC) of the present invention support replication of all the native HCV infectious clones that are predominantly represented in HCV infected individuals. Lindenbach et al., were only able to replicate molecular clones (full length chimera) of HCV derived from a Japanese fulminant hepatitis patient (it is called JFH-1 strain). The JFH-1 strain of HCV is uniquely virulent. For unexplained reasons, it is the only strain of HCV that grows in a variety of cell lines without culture adaptive mutations.
(b) Each native strains of HCV display different rates of viral RNA replication and sensitivity to interferon treatment (the only approved therapy for HCV chronic hepatitis). As proof of principle, our HCV replication system recapitulates the characteristic differences in rates of growth and the sensitivities to interferon treatment of HCV genotypes 1a, 1b and 2a, seen in natural infections. That is, HCV 1a which is the fastest growing and least responsive to interferon therapy in natural infections, behaves similarly when grown in our primary hepatocyte cultures.

None of these native properties of natural HCV infection was reported by Lindenbach et al., in their system they were able to replicate molecularly derived hybrid clones of only the JFH-1 strain of HCV, which as mentioned above, is unusually virulent. Importantly, their culture system utilized hepatoma cell line Huh-7.5, which as described by Sumpter et al., (2005) suffers inactivating mutation in RIG-I gene that renders it unusually permissive for viral replication. It is important to note that unlike our primary hepatocyte culture system, host cells that lack RIG-I function will be unlikely to mount natural host response to virus infections. Thus our system is inherently more suitable for antiviral drug discovery and vaccine against HCV.

The HCV permissive human primary liver cell system of the invention greatly facilitates the skilled artisan's ability to test more effective therapies against chronic hepatitis and hepatocellular carcinoma.

### BRIEF DESCRIPTION OF THE DRAWINGS

FIG. 1. H&E staining of tranverse section of sixty-day culture of human hepatocytes mass, showing features of “normal” liver such as organized hepatocytes with defined nucleus and some binucleated cells (A and B). Immunohistochemical staining of Factor VIII for sinusoidal endothelial cells (C) shows the formation of a central hepatic venule.

FIG. 2. RT-PCR results of RNA extracted from hepatocyte culture showing (as indicated) expression of albumin, alpha Feto-Protein, cytokeratin 18 and TGF-β (indicators of normal physiological function of human hepatocytes), in 30 day cultures. Lanes 1 and 2 are RNA samples from two different cultures. Lane M represents a molecular marker.

FIG. 3. Br-UTP labeled HCV RNA: (A) Normal hepatocytes treated with 5 μg/ml Actinomycin D for 30 minutes prior to 45 minute Br-UTP (25 μM) incubation at 37° C., followed by two 15 minute ‘chases’ with fresh HDM medium supplemented with 5% FBS. Similar age hepatocyte cultures were transfected with, (B) a wild type HCV replicon, or (C) the Polymerase mutant HCV, and on the third day post-transfection RNA was labeled with Br-UTP in the presence of Actinomycin D as in (A). (D) β-THP-1 cells expressing DC-SIGN were co-cultured with human hepatocytes 24 hours before transfection with wild type HCV replicon and on the third day post-transfection HCV RNA was labeled with Br-UTP as in (A). Immunofluorescence of Br-UTP labeled RNA in each case was detected with Texas red conjugated anti-Br-DU antibody.

FIG. 4. The confocal image shows the co-localization of nascent viral protein (green) and RNA (red) in hepatocytes transfected with pl/5A-GFP (wild-type) HCV replicon (lower panel). Br-UTP labeling of HCV RNA is shown in red and HCV protein in green in the cytoplasmic compartment; both were enhanced in the presence of co-cultured DC-SIGN expressing cells (upper panels).

FIG. 5A. FIG. 5 A illustrates results of primary cultures transfected with 1 μg of run-off transcripts of full length HCV genotype 1a (pCV-H77c), genotype 1b (pCV-J4L6S) or genotype 2a (pJ6CF) by FuGene 6 lipofection procedure. Viral RNA was RT-PCR amplified from total cell RNA six days post-infection.

FIG. 5B. FIG. 5B illustrates HCV infection of human primary hepatocytes with (1 ml) virus suspension from filtered culture media (shown in 5A). Viral RNA replication was determined at six day post-infection (as in 5A).

FIG. 6A: Shows CD81 dependence of HCV infection. Hepatocytes pre-incubated with monoclonal antibody against CD81 HCV co-receptor, showed markedly reduced infection with HCV genotype 1a.

FIG. 6B. FIG. 6B illustrates inhibition of HCV replication by IFN-alpha (R&D system) treatment. Primary hepatocytes infected with the HCV genotypes were treated with indicated concentrations of IFN and the viral RNA was amplified from total cell RNA six days post-infection (as in 5A).

### SUMMARY OF THE INVENTION

This invention relates to long-term culture of human primary hepatocyte systems useful for culture of human hepatotrophic and other viruses, in particular human hepatiis C virus (HCV), replication, expression and dissemination. The invention also relates to methods for determining the cellular response to virus, particularly HCV, replication, and dissemination in hepatocytes. The invention further relates to methods for determining the effectiveness of pharmaceuticals useful in both protective and therapeutic treatment of parasite and virus, particularly HCV, infection. The invention further relates to efficient propagation of the infectious clones of HCV genotypes 1a, 1b, and 2a which are predominant forms of HCV infections in patients in the U.S.

The present invention relates to a long-term human hepatocyte culture system derived from normal, untransformed, primary human hepatocytes, wherein the culture system is permissive to propagate all strains of native hepatitis C virus. According to the present invention, the long-term human hepatocyte culture system can be maintained indefinitely, revived periodically from frozen stocks (saved in liquid nitrogen). The long-term human hepatocyte culture system of the present invention is sensitive to drugs that inhibit growth of hepatitis C virus.

The present invention also relates to a marker for hepatitis C virus caused disease progression from a cell culture system that support its growth, wherein the culture system is permissive to propagate about 100% of genotype 1a of hepatitis C virus. The marker of the present invention provides indicators for designing more accurate or effective treatments for liver diseases. The marker of the present invention also provides indicators for designing more accurate or effective treatments for hepatitis C virus. In addition; the marker of the present invention provides indicators for designing more accurate or effective treatments for liver cancer.

### DETAILED DESCRIPTION OF THE INVENTION

Hepatocytes are primary targets of hepatitis C virus (HCV) infection. An efficient system of native HCV replication would greatly facilitate development of antiviral drugs and vaccine against HCV infection, which afflicts nearly 175 million people worldwide. The invention describes a replication competent primary hepatocyte culture system useful for understanding the regulation of viral replication, expression and dissemination in HCV infected human liver. Studies were designed to ask: (i) whether the kinetics of HCV replication in primary liver cell culture supports co-localization of native viral proteins and HCV RNA; (ii) whether the nascent HCV proteins and RNA assemble into infectious virus particles; and (iii) whether HCV virus dissemination in human primary liver cells is dependent on cellular co-receptor CD81, and the ‘trans-receptor’ proteins DC-SIGN an L-SIGN. These studies define at least three milestones: (i) to analyze the biochemical and morphologic criteria of native HCV particles synthesized in primary liver cells, (ii) to ascertain the requirements cellular proteins in HCV dissemination, and (iii) to determine the contributions of human primary liver cell types in initiating and sustaining HCV infection. The knowledge gained from these studies significantly enhances the understanding of the stages of HCV infection and dissemination in human liver, and helps designing and testing novel treatments for chronic hepatitis and hepatocellular carcinoma.

### BACKGROUND AND SIGNIFICANCE

Lack of suitable cell culture system for efficient replication of native HCV has been, a significant handicap in our attempt to study factors that regulate viral dissemination in human liver. Primary hepatocytes in human liver cultures may be distinct from isolated hepatocyte cell lines in their ability to sustain HCV replication, since they retain normal functions of key antiviral response genes. Contributions of other nonparenchymal cell types in primary liver cell cultures, lacking in hepatoma cell lines, for example, liver sinusoidal endothelial cells (LSECs) that express cell surface lectins, L-SIGN which bind to HCV envelop glycoproteins E1 and E2, likely enhance the chances of native HCV replication. Analogous to the antigen presenting dendritic cells, Kupffer cells in liver are known to produce cell surface lectins, DC-SIGN with strong affinity for HCV envelope glycoproteins. Recent studies with HIV/HCV pseudotyped particles bearing HCV glycoproteins E1/E2 suggest that the cell surface lectins function as ‘trans-receptor’ in HCV dissemination.

These studies are designed on the premise that efficient replication of native HCV virus may require cooperative interaction of cell types accessible in primary liver cultures, that are lacking in isolated cell lines. The invention provides for efficient HCV replication in human primary liver cultures. The invention allows one to reach at least three milestones:

1. To show that the native HCV proteins and RNA co-localize in and proliferate in hepatocytes; and that the dissemination of HCV is dependent on host cell co-receptor proteins.

2. To characterize biochemical assembly of infectious HCV particles synthesized in primary hepatocytes that are capable of spreading infection.

3. To evaluate the molecular basis of replication competent phenotype by quantitative proteome analysis.

HCV is an RNA virus of the family Flaviviridae. Six major genotypes of naturally occurring variants of HCV have been classified. These include 1a (pCV-H77c), 1b (pCV-J4L6S), 2a (pJ6CF), 2b 3 and 4. The HCV genotypes 2, 3 and 4 are represented in less than 10% of HCV infected people. These last three HCV genotypes show rarely in HCV infected patients in the USA. Inherently slow rates of growth, of any HCV genotype has less to do with any ‘mutations’, but may be more a function how well each strain is able of evading human innate antiviral defenses. The native virus replication in the primary cell culture system of the present invention which retains the normal host response genes will be more suitable for identifying potent antiviral drugs and candidate proteins for developing vaccine against HCV infection.

The culture system of human primary hepatocytes of the present invention can be utilized to replicate full length infectious native clones of HCV. Run off transcripts of full length HCV genotypes, such as 1a (pCV-H77c), 1b, (pCV-J4L6S) and 2a (pJ6CF), can be introduced into HHC cultures by lipofection, and viral RNA can be RT-PCR amplified from infected cells via the culture system of the present invention. The difference in the rates of replication of the native HCV genotypes is more a reflection of their inherent replication properties, as mentioned above. The inventors of this invention initiate each culture with equal amounts of full length RNAs of the infectious clones.

The culture system of the present invention can be made by overlaying purified human primary hepatocytes on a feeder layer of cells for optimal attachment. The cocultures are maintained in hormonally defined medium without serum. The system is easily adaptable to culturing hepatocytes from individual donors, and thus suitable for the analysis of broader array of hepatotropic pathogen.

Vaccines and markers for liver diseases; including HCV can be developed via the cell culture system of the present invention. The rationale for the markers is that as the disease progresses, human host attempts to fight off the pathogen by raising antibodies against abnormal proteins that are made as a consequence of viral infection. Since the invention of the culture system, for the first time, faithfully replicates native virus, any altered forms of the proteins during the course of HCV replication can be identified by its specific binding to the antibodies from HCV infected patients (the so called antigen-antibody interaction). The inventors of the present invention ‘display’ the total protein from HCV infected human primary cells on the surface of recombinant bacteriophage (the so called phage-display libraries), and purify individual clones by iterative bio-panning the phage library with patient antibody IgG.

The individual protein coding genes from the phage-display library is then sequenced and characterized as prognostic markers for HCV mediated liver cirrhosis and hepatocellular carcinomas. These marker proteins, the so called ‘eptitopes’ can be developed as candidates for vaccine development based on their properties to neutralize HCV propagation.

Methods:

Primary Culture of Human Hepatocytes

Freshly isolated human hepatocyte cell suspension was obtained from Cambrex Bioscience Inc. (Walkersville, Md., along with relevant clinical information on the donor tissue). After determining cell viability by trypan blue exclusion, the hepatocytes were plated on a semi-confluent monolayer of a hepatic stellate, feeder cell line for efficient cell attachment (Arnaud, et al., 2002), in Modified Eagle's Medium (MEM) containing 5% Fetal Bovine Serum (FBS), insulin (50 mM), 1% essential amino acids (100×) and penicillin/streptomycin (100 mg/L). Following incubation at 37° C. in 5% CO₂ for 3 hours, media was replaced by a hormonally defined medium (HDM) comprised of: albumin (2 g/L), glucose (2 g/L), galactose (2 g/L), ornithine (0.1 g/L), proline (0.03 g/L), nicotinamide (0.610 g/L), ZnCl₂ (0.544 mg/L), ZnSO4 (0.75 mg/L), CuSO₄ (0.2 mg/L), MnSO₄ (0.025 mg/L), glutamine (5 mM), ITS (Insulin transferrin. sodium selenite; 1 g/L), dexamethasone (10⁻⁷ M), TGF α (20 ng/mL), 1% essential amino acids (100×) and penicillin/streptomycin (100 mg/L). During long-term culture, media was replaced by fresh HDM every 48 hours. Within four weeks, the hepatocyte culture forms vertical growth (FIGS. 1A and B show histological sections through a sixty day hepatocyte culture). The feeder cells, which require FBS for growth, were attenuated during long term culture in HDM medium (which lacks FBS). All subsequent studies were carried out on hepatocytes revived from frozen stocks of sixty-day culture.

Histology of Human Liver Culture

Paraffin embedded tissue sections (5 micron thick) were deparaffinized and dehydrated by incubating successively with increasing concentrations of ethanol and stained in Harris hematoxylin solution (Sigma) for 30 minutes. Stained sections were rinsed in tap water and dipped 6 times in differentiating solution, then dipped in 95% ethanol for two minutes. The sections were stained in alcoholic Eosin Y (Sigma) for 20 minutes and washed in 100% ethanol 5 times for 3 minutes each, cleared in xylene and mounted under a cover slip using Gel/mount (Biomeda Corp.). Sinusoidal endothelial cells were stained (using the Vectastain protocol) with factor VIII primary antibody (Vector Labs) and biotinylated secondary anti-rabbit anti-body (Vector Labs) (FIG. 1 C).

RT-PCR

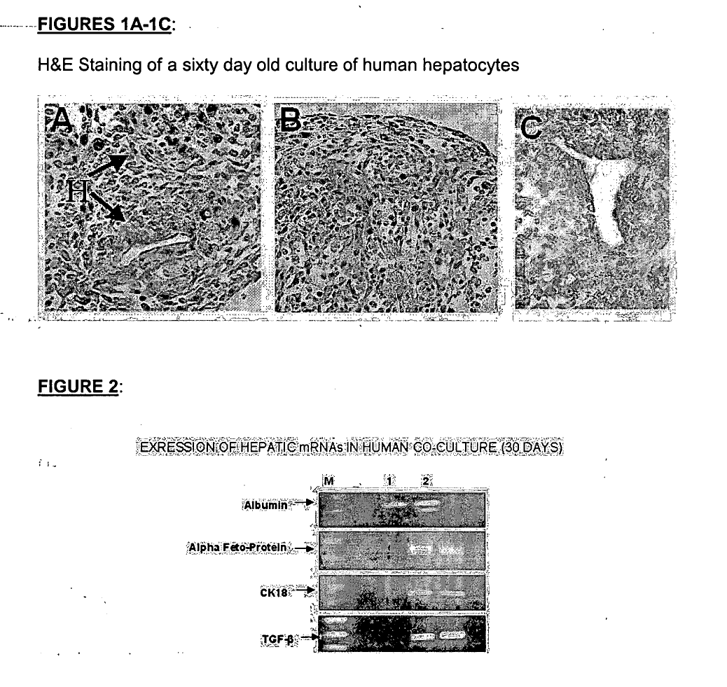

To characterize the biochemical markers for hepatocyte function, the RNA samples from 30-day and 60-day old cultures were analyzed for the levels of expression of (i) albumin, (ii) alpha-fetoprotein, (iii) Cytokeratin 18, (iv), TGF-β1. FIG. 2 shows results using a 30-day culture. RNA was isolated using TRIzol LS reagent (Invitrogen) as per manufacturer's protocol. The precipitated RNA was dissolved in RNase-free water by incubating for 10 minutes at 60° C. and stored at −80° C. until use.

The following PCR cycle conditions were established (using Gene Amp PCR system 9700 PE; Applied Biosystems). The conditions used were: 1 cycle at 55° C. for 30 minutes and denaturation at 94° C. for 2 minutes; 35 cycles of amplification at 94° C. for 15 seconds, annealing at 60° C. for 30 seconds, and extending at 72° C. for 7 min. The reaction was carried out in a 50 μl reaction volume containing 25 μl of 2× Reaction Mix, 1 μg of template RNA, 0.2 μM each of sense and anti-sense primers, 1 μl of RT/Platinum® Taq Mix, and RNase-free distilled water to 50 μl. The amplification products were analyzed on a 2% agarose gel.

Expression of HCV Replicons in Human Hepatocytes

(i) Protein Expression

The assays for replication competent HCV was based on nascent viral RNA synthesis. The nascent viral protein was monitored by western blotting with specific HCV antibodies. Runoff transcripts were prepared in vitro and introduced into culture cells by lipofection with FuGENE-6 (Roche) as per manufacturer's protocol. One microgram each of the wild type and polymerase mutant transcript was transfected into each six well plate. Maximum efficiency was obtained using a transfection ratio of 1 μg of HCV RNA and 6 μl of FuGene-6 in serum free medium (in a final transfection mixture volume of 100 μL). Twenty four hours after transfection, the medium was replaced with freshly made defined growth media and the cells were allowed to grow for the indicated time period.

(ii) RNA Replication

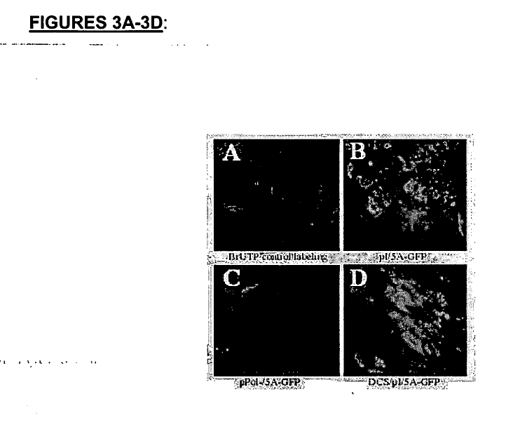

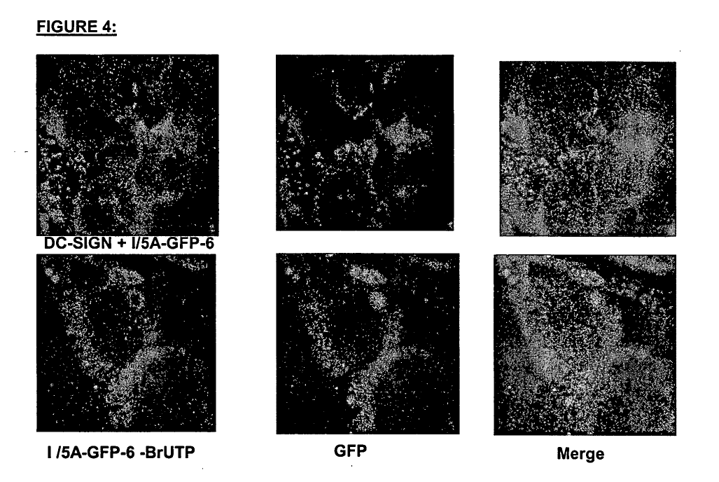

To determine the replication of HCV RNA, infected cells were metabolically labeled with 5-bromouridine-5′-triphosphate (Br-UTP) (FIG. 3). The protocol for Br-UTP-labeling was as follows: hepatocyte cultures were grown in 6 well plates and were transfected with HCV RNA using FuGENE 6 as described above. The cells were incubated with actinomycin D (5 mg/ml) for 30 min to inhibit cellular DNA-dependent RNA polymerases. To introduce Br-UTP into the cells, 6 μl FuGENE 6 reagent was mixed with 89 μl of Opti-MEM and incubated for 5 min at RT. Five microliters of Br-UTP (100 mM) was added to the FuGENE 6 mixture and incubated for 15 minutes at room temperature. Following incubation for 45 minutes at 37° C. (in 5% CO₂), the unincorporated Br-UTP was chased with fresh growth media containing 5% FBS and HDM, replaced twice for 15 minutes each. The cultures were then washed with PBS and fixed with 4% paraformaldehyde (at room temperature for 10 minutes), and rinsed twice with PBS, for 5 minutes each. Free formaldehyde groups were blocked with 0.2% glycine for 10 minutes. The cells were then permeabilized with 0.5% Triton X-100 in PBS for 5 minutes at room temperature and washed with PBS twice. Cells were then treated with 5% normal donkey serum in PBS for 40 minutes at 4° C. to avoid non-specific binding of immunoglobulins, and incubated with primary antibody anti-bromodeoxyuridine mouse IgG monoclonal PRB-1 (Molecular Probes, 1:500 dilution) overnight at 4° C. followed by three washes and treatment with secondary Alexa Fluor 568 goat anti-mouse IgG (H+ L) conjugated to Texas red (Molecular Probes, 1:80) for 1 hour at room temperature. Br-UTP labeled HCV RNA in cells transfected with wild type replicons is markedly induced (FIG. 3B). Considering that the Br-UTP incorporation into nascent HCV RNA (shown in FIG. 3) represents a 45 minute ‘pulse’ labeling of cells (three day post-transfection with HCV replicons), the results suggest a strong rate of HCV replication in human primary hepatocytes. Next, whether the nascent viral proteins and RNA co-localized in similar cellular compartments was determined by confocal microscopy. HCV proteins (green punctuate cytoplasmic structures, FIG. 4, panels B and E), and the RNA (red, FIG. 4, panels A and D), overlapped as orange cytoplasmic granules (FIG. 4, panels C and F) by confocal microscopy (the scanning laser microscope equipped for 476-nm (FITC) and 529-nm (Cy5) and 586-nm (Texas red).

(iii) Propagation of Native HCV in Human Primary Liver Cells

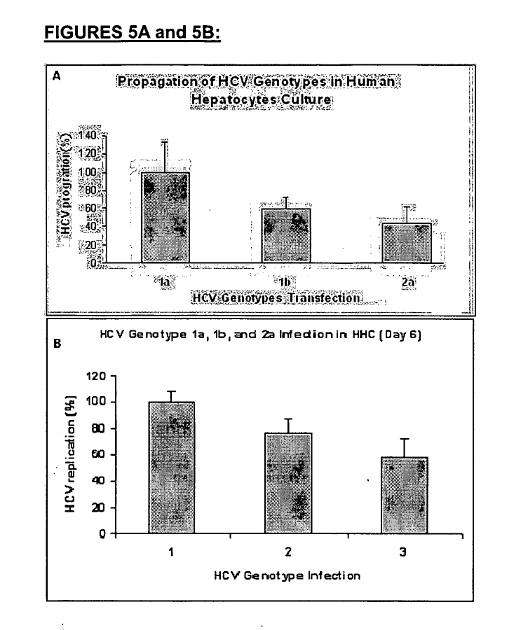

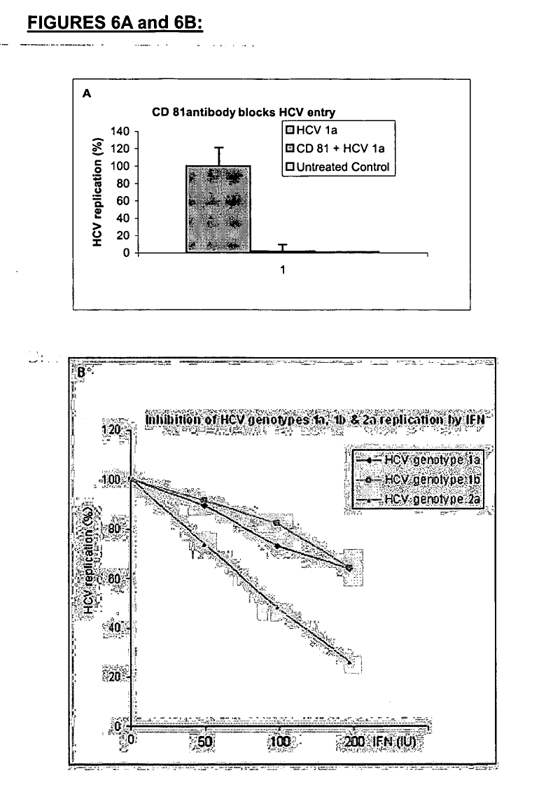

Replication of Infectious clones of HCV: We determined HCV permissive properties of human primary hepatocyte cultures by introducing full length genomic clones of HCV genotypes 1a, 1b and 2a into the culture system by transfection methods outlined above. As shown in FIG. 5A, cultures transfected with full length genomic clones of the HCV genotypes showed differences in their rates of replication; genotype 1a being the fastest replicating strain. We next determined if the propagation of HCV was dependent on viral interaction with CD81 co-receptor. Cultures pre-incubated with monoclonal antibody against CD81 (FIG. 6A) showed complete block of HCV replication (data from only HCV genotype 1a replication is shown).

Next important issue is whether our primary culture system for replicating full length HCV clones produces infectious HCV. We tested the presence of infectious virus in the culture media from cells transfected with the HCV genotypes (shown in FIG. 5A). When the virus stocks from filtered culture media was used to inoculate naïve cultures, the rate of HCV replication (FIG. 5B) with respect to the three HCV genotypes was similar to cells transfected with the full length genomic clones (FIG. 5A).

In natural infections patients with HCV genotype 1 infection are less responsive to IFN therapy than for example, HCV genotype 2a infections. We reasoned that the relative sensitivities of the various HCV strains to IFN therapy may be related to the virus mediated down regulation of host cell immunity. Since we are able to replicate native strains of HCV in our in vitro system, we next asked whether HCV replication shows differential sensitivities to inhibition by IFN alpha (FIG. 6B). As shown in FIG. 6B, HCV genotype 1 is far less responsive to IFN treatment than is HCV genotype 2a, analogous to the experience in clinical setting. Overall, these results suggest that our culture system of primary hepatocytes is optimal for the replication of native HCV virus; and the host response to viral infection recapitulates natural infection in human populations. Thus our approach to propagate native HCV in human primary liver cell cultures appears uniquely suited for the evaluation of potent antiviral drugs and candidate vaccine against HCV infection.

### DETAILED DISCUSSION OF FIGURES

Replication of full length, infectious clones of HCV genotypes 1a, 1b & 2a: An important aspect of the human primary hepatocyte system is that it should be able to support sustained replication of infectious clones of HCV. We show the proof of principle by introducing full length genomic clones of HCV into the primary hepatocyte cultures and observe virus replication for a period of time, up to a week. Primary hepatocytes were transfected with full length run-off transcripts of the HCV genotypes (using FuGENE 6 lipofection technique describes above). Total cell RNA was prepared six days post-transfection, and the HCV RNA was amplified with nested RT-PCR using primers that specifically amplify viral RNA from the infected cell total RNA. Results from three independent transfections is illustrated (FIG. 5A) as relative rates of replication (HCV genotype 1a as 100%, and HCV genotypes 1b and 2a as 73% and 53% of 1a respectively).

A crucial test of the human primary culture system we describe is whether it will produce infectious HCV virus. Infectivity of the HCV genotypes 1a, 1b and 2a was determined with filtered culture media from cells transfected with full length HCV genomic clones (described above, FIG. 5A). The test of the culture system we describe, is that it should replicate infectious virus particles; and the infectious virus should be recovered from the culture media. That is, the virus particle recovered from the culture media should, upon inoculation of naïve cells, be able to initiate spreading infection of HCV. We challenged fresh human hepatocyte cultures with 1 ml filtered supernatant from HCV transfected cultures (described in FIG. 5A, above). The propagation of infectious HCV was monitored by analyzing viral RNA, RT-PCR amplified as before, six-day post infection from total cell RNA (results shown in FIG. 5B represent three independent infections). There are two important points to be made from these results: (i) that HCV virus produced in the human primary culture system we describe is capable of infecting naïve cells; and (ii) the sustained propagation and the rates of replication of the three HCV genotypes is comparable to natural infection. This important result, for the first time, demonstrates that the propagation of native HCV can be reproduced in our culture system.

An important test of our human primary liver cell culture system is that the replication of native HCV should be sensitive to its binding to the known host cell receptor. And importantly, native HCV replication in the primary culture system should recapitulate the response to therapy observed in natural infections. As we show (the results in FIG. 6), our human primay hpatocyte system meets both these conditions. Antibody blocking of the cellular receptor, CD81, (which HCV requires for attachemnet to the host), prior to virus infection, completely eliminates virus replication (FIG. 6A). In the test for IFN therapy, when we treat the HCV infected cells with increasing concentrations of interferon alpha (results shown in FIG. 6B), the replication of HCV is inhibited in parallel with the observation in natural infections. That is, HCV genotype 1 infection is far less sensitive inhibition by IFN than is HCV genotype 2a, similar to the experience of HCV infected patients. The important lesion from these experiments is that the human primary hepatocyte culture not only sustains native HCV replication, it also recapitulates the sensitivity of the virus to an approved IFN therapy. In total these studies confirm that the human primary culture system for the propagation of HCV we describe, is complete in its ability to replicate infectious virus and its sensitivity to therapy. This makes the human primary hepatocyte culture system uniquely suited for the development and testing of new antiviral therapy and vaccine against HCV infection. HCV entry is dependent on CD81: Culture were pre-blocked with 10 ug/ml mAb CD81 (Santa Cruz) prior to transfection with HCV genotype 1a. Viral RNA replication was determined six days post-transfection as in 5. (B). Inhibition of HCV replication by IFN-alpha (R&D system) treatment. Primary hepatocytes infected with the HCV genotypes were treated with indicated concentrations of IFN and the viral RNA was amplified from total cell RNA six days post-infection (as in FIG. 5).

Exemplary Diseases

The invention, as exemplified for HCV above, is useful for the characterization of, and treatment and pharmaceutical development against, any disease that affects the liver. Such diseases include:

Alagille Syndrome, Alpha 1—Antitrypsin Deficiency, Autoimmune Hepatitis, Biliary Atresia, Chronic Hepatitis, Cancer of liver, Cirrhosis, Cystic Disease of the liver, Fatty Liver, Galactosemia, Gallstones, Gilbert's Syndrome, Hemochromatosis, Hepatitis A, B and C, Neonatal Hepatitis, Porphyria, Primary Biliary Cirrhosis, Primary Sclerosing Cholangitis, Reye's Syndrome, Sarcoidosis, Tyrosinemia, Type I Glycogen Storage Disease and Wilson's Disease.

Additionally, the invention is useful for the study of, and treatment and pharmaceutical development against, any parasite that infects or passes through the liver. Such parasites include:

E. multilocularis, Echinococcus vogeli, Schistosoma spp. Fasciola hepatica, Clonorchis sinensis, Opisthorchis viverrini, Opisthorchis felineus, Cryptosporidium parvum, Entamoeba histolytica, Echinococcus granulosus

Leishmania sp, Enterocytozoon bieneusi, Plasmodium falciparum, Baylisascaris procyonis.

As indicated above, the invention is also useful in the development and testing of pharmaceutical compounds and compositions. Under this aspect of the invention, various compounds and compositions are applied to the cell culture and their effect on a disease or infection determined (in the case of a parasitic disease, normally by challenge with the parasite). Alternatively, for parasites, the cells may first be infected, and then the pharmaceutical compound or composition then applied. The invention, as such, provides a useful means to rapidly screen many pharmaceuticals for their potential therapeutic effectiveness.

### INCORPORATION BY REFERENCE

Appended hereto, as a part of the invention disclosure, is APPENDIX I (three pages) of publications cited herein. The publications cited in APPENDIX I are incorporated herein by their reference hereto.

It is well understood that various other modifications will be apparent to, and can readily be made by those skilled in the art without departing from the scope and spirit of this invention. Accordingly, it is not intended that the scope of the invention be limited to the description set forth above, but rather be construed as encompassing all of the features of patentable novelty which reside in the present invention, including all features which would be treated as equivalents thereof by those skilled in the art to which the invention pertains.

### APPENDIX I

### REFERENCES

Agnello, V., Able, G., Elfahal, M., Knight, G. B., and Zhang, Q. X. (1999) Flavoviridae viruses enter cells via low density lipoprotein receptor. Proc. Natl. Acad. Sci. USA 96:12766-12771.
Alter, H. J. and Seef, L. B. (2000) Recovery, persistence, and sequelae in hepatitis C virus infection: a perspective on long-term outcome. Semin. Liver Dis. 20:17-35.
Alvarez, C. P., Lasala, F., Carrillo, J., Muniz, O., Corbi, A. L., and Delgado, R. (2002). C-type lectins DC-SIGN and L-SIGN mediate cellular entry by Ebola virus in cis and trans. J. Virol. 76: 6841-6844.
Arnaud, A., Fontana. L., Saez-Lara, M. J., Gil, A., Lopez-Pedrosa, J. M., (2002) Exogenous nucleosides modulate the expression of rat liver extracellular matrix genes in single cultures of primary hepatocytes and a liver stellate cell line and in their co-culture. J Hepatol. 2002 May;36(5):590-9.
Boisvert, J., He, X. S., Cheung, R., Keeffe, E. B., Wright, T., and Greenberg, H. B. (2001). Quantitative analysis of hepatitis C virus in peripheral blood and liver: replication detected only in liver. J. Infect. Dis. 184: 827-835.
Cormier, E. G., Durso, R. J., Tsamis, F., Boussemart, L., Manix, C., Olson, W. C., Gardner, J. P. and Dragic, T. (2004). L-SIGN and DC-SIGN mediate transinfection of liver cells by hepatitis C virus. Proc. Natl. Acad. Sci. USA. 101: 14067-14072.
Cormier, E. G., Tsamis, F., Kajumo, F., Durso, R. J., Gardner, J. P., and Dragic, T. (2004a). CD81 is an entry coreceptor for hepatitis C virus. Proc. Natl. Acad. Sci. USA. 101: 7270-7274.
Flint, M., Logvinoff, C., Rice, C. M., and McKeating, J. A. (2004). Characterization of infectious retroviral pseudotype particles bearing hepatitis C virus glycoproteins (2004). J. Virol. 78:6875-6882.
Gale, M., Foy E. M., (2005) Erratum: Evasion of intracellular host defence by hepatitis C virus. Nature; 437:290.
Geijtenbeek, T. B., Kwon, D. S., Torensma, R., van Vliet, S. J., et al., (2000). DC-SIGN, a dendritic cell-specific HIV-1-binding protein that enhances transinfection of T cells. Cell 100: 587-597.
Lagging, L. M., Meyer, K., Owens, R. J., and Ray, R., (1998). Functional role of hepatitis C virus chimeric glycoproteins in the infectivity of pseudotyped virus. J. Virol. 72: 3539-3546.
Linda, B. Jacobsen, Susan, A. Calvin, Kim, E. Colvin and MaryJo, Wright., (2004). FuGENE 6 Transfection Reagent: the gentle power. Methods. 33:(2), 104-112
Lindenbach, B. D., Evans, M. J., Syder, A. J., Wolk, B., Tellinghuisen, T. L., Liu C. C., Maruyama, T., Hynes, R. O., Burton, D. R., McKeating, J. A., Rice, C. M., (2005) Complete replication of hepatitis C virus in cell culture. Science. July 22;309(5734):623-6. Epub 2005 Jun. 9.
Lozach, P-Y., Amara, A., Bartosch, B., Virelizier, J-L., et al., (2004). C-type lectins L-SIGN and DC-SIGN capture and transmit infectious hepatitis C virus pseudoparticles. J. Biol. Chem. 279:32035-32045.
Ludwig, I. S., Lekkerkerker, A. N., Depla, E., Bosman, F., Musters, R. J. P., et al., (2004). Hepatitis C virus targets DC-SIGN and L-SIGN to escape lysosomal degradation. J. Virol. 78: 8322-8332.
Moradpour, D., Evans, M. J., Gosert, R., Yuan, Z., Blum, H. E., Goff, S. P., Lindenbach, B. D., and Rice, C. M., (2004). Insertion of green fluorescent protein into nonstructural protein 5A allows direct visualization of functional hepatitis C virus replication complexes. J. Virol. 78: 7400-7409.
Pileri, P., Uematsu, Y., Campagnolli, S., Galli, G., Falugi, F., et al., (1998) Binding of Hepatitis C Virus to CD81. Science 282: 938-941.
Pohlmann, S., Zhang, J., Baribaud, F., Chen, Z., Leslie, G. J., et al., (2003). Hepatitis C virus glycoproteins interact with DC-SIGN and DC-SIGNR. J. Virol. 77: 4070-4080.
Sumpter, R. Jr, Loo, Y. M., Foy, E., Li. K., Yoneyama. M., Fujita, T., Lemon. S. M., Gale. M. Jr., (2005) Regulating intracellular antiviral defense and permissiveness to hepatitis C virus RNA replication through a cellular RNA helicase, RIG-I., J. Virol. March;79(5):2689-99.
Wakita. T., Pietschmann, T., Kato, T., Date, T., Miyamoto. M., Zhao. Z., Murthy, K., Habermann, A., Krausslich, H. G., Mizokami, M., Bartenschlager, R., Liang, T. J. Production of infectious hepatitis C virus in tissue culture from a cloned viral genome. Nat Med. 2005 July;11(7):791-6. Epub 2005 Jun. 12. Erratum in: Nat Med. 2005 August; 11(8):905.
Yanagi, M., Purcell, R. H., Emerson, S. U., Bukh, J., (1997) Transcripts from a single full-length cDNA clone of hepatitis C virus are infectious when directly transfected into the liver of a chimpanzee. Proc Natl Acad Sci USA. August 5;94(16):8738-43.
Yoneyama, M., Kikuchi, M., Natsukawa, T., Shinobu, N., Imaizumi, T., Miyagishi, M., Taira, K., Akira, S., Fujita, T., (2004) The RNA helicase RIG-I has an essential function in double-stranded RNA-induced innate antiviral responses. Nat Immunol July;5(7):730-7. Epub 2004 Jun. 20.
Zhang, J., Randall, G., Higginbottom, A., Monk, P., Rice, C. M., and Mckeating, J. A., (2004). CD81 is required for hepatitis C virus glycoprotein-mediated viral infection. J. Virol. 78: 1448-1455.
Zhong, J., Gastaminza, P., Cheng, G., Kapadia, S., Kato, T., Burton, D. R., Wieland, S. F., Uprichard, S. L., Wakita. T., Chisari, F. V., (2005) Robust hepatitis C virus infection in vitro. Proc Natl Acad Sci USA. June 28;102(26):9294-9. Epub 2005 Jun. 6.

We claim:

1. A long-term human hepatocyte culture system derived from normal, untransformed, primary human hepatocytes, wherein the culture system is permissive to propagate all native hepatitis C virus.

2. The long-term human hepatocyte culture system according to claim 1, wherein said culture can be maintained indefinitely revived periodically from frozen stocks.

3. The long-term human hepatocyte culture system according to claim 1, wherein the culture is sensitive to drugs that inhibit growth of hepatitis C virus.

4. A marker for hepatitis C virus caused disease progressed from a cell culture system that support its growth, wherein the culture system is permissive to propagate about 100% of genotype 1a of hepatitis C virus.

5. The marker according to claim 4, wherein the marker provides indicators for designing more accurate or effective treatments for liver diseases.

6. The marker according to claim 4, wherein the marker provides indicators for designing more accurate or effective treatments for hepatitis C virus.

7. The marker according to claim 4, wherein the marker provides indicators for designing more accurate or effective treatments for liver cancer.
